(19) United States
(12) Patent Application Publication
(10) Pub. No.: US 2026/0335583 A1
(43) Pub. Date: Oct. 29, 2026

(54) ENDOTRACHEAL TUBE HOLDING DEVICE

(71) Applicant: Hadassah Sodden, Pearl River, NY (US)

(72) Inventor: Hadassah Sodden, Pearl River, NY (US)

(21) Appl. No.: 19/636,129

(22) Filed: Apr. 1, 2026

Related U.S. Application Data
(60) Provisional application No. 63/795,828, filed on Apr. 28, 2025.

(51) Int. Cl. A61M 16/06

(52) U.S. Cl. CPC A61M 16/0683

(57) ABSTRACT

The device is configured to stabilize and retain an endotracheal tube relative to a patient using structural support and adjustable retention components. A posterior strap extends around occipital and cervical regions to distribute retention forces and maintain positioning. Opposing ends of the posterior strap are coupled to cheek support members having elastomer gel padding configured to conform to facial anatomy and provide releasable adherence with controlled shear resistance. A head strap extends across a superior region of the head to provide vertical stabilization. A connection member extends between the cheek support members across an upper lip region and supports a tube receiving housing while maintaining spacing from oral and nasal structures. The tube receiving housing is translatable along the connection member through complementary fastener interfaces to permit positional adjustment and includes a receiving port configured to engage an outer surface of the endotracheal tube.

### CROSS-REFERENCE TO RELATED APPLICATION

The present application claims priority to, and the benefit of, U.S. Provisional Application No. 63/795,828, which was filed on Apr. 28, 2025, and is incorporated herein by reference in its entirety.

### FIELD OF THE INVENTION

The present invention relates generally to the field of endotracheal tubes. More specifically, the present invention relates to a device configured to stabilize and retain an endotracheal tube relative to a patient. Accordingly, the present disclosure makes specific reference thereto. Nonetheless, it is to be appreciated that aspects of the present invention are also equally applicable to other like applications, devices, and methods of manufacture.

### BACKGROUND

Adhesive-based stabilization systems for airway management devices present limitations in many clinical environments. More specifically, patients who have facial hair often experience reduced adhesion from said systems, which can compromise the securement of an endotracheal tube holding device. Inadequate stabilization increases the risk of unintended tube displacement during patient movement, repositioning, or routine care procedures. Prolonged use of adhesive materials may also contribute to skin irritation, pressure-related injuries, and formation of sores. These complications can lead to discomfort, delayed recovery, and additional clinical interventions. Inconsistent tube retention may result in accidental extubation or airway trauma, both of which can pose significant risks to patient safety. Clinical staff therefore require alternative securement methods that maintain stability while minimizing adverse skin effects.

Therefore, there exists a long-felt need in the art for a device that securely stabilizes an endotracheal tube. There also exists a long-felt need in the art for an endotracheal tube holding device that reduces skin irritation and pressure-related breakdown during prolonged use. Moreover, there exists a long-felt need in the art for an endotracheal tube holding device that accommodates patients with facial hair while maintaining adjustable positioning and comfort.

The subject matter disclosed and claimed herein, in one embodiment thereof, comprises an endotracheal tube holding device. The device is configured to stabilize and retain an endotracheal tube relative to a patient through a combination of structural support members and adjustable retention components. The device may comprise a posterior strap extending around the occipital and cervical regions to distribute retention forces and maintain positioning during clinical use. Opposing ends of the posterior strap may be coupled to cheek support members having mineral-based elastomer gel padding layers configured to conform to facial anatomy and provide releasable adherence with controlled shear resistance. A head strap may extend between the cheek support members across a superior region of the head to provide vertical stabilization and accommodate varied patient orientations. A connection member may extend between the cheek support members across an upper lip region to support a tube receiving housing while maintaining spacing from oral and nasal structures. The tube receiving housing may be translatable along the connection member through complementary fastener interfaces configured to permit incremental or continuous positional adjustment. The tube receiving housing may further comprise a receiving port having an inner fastener configured to engage an outer surface of the endotracheal tube. Padding elements positioned along patient contacting surfaces may further enhance comfort and reduce localized pressure during extended use.

In this manner, the endotracheal tube holding device of the present invention accomplishes all the foregoing objectives and provides a device configured for secure stabilization of an endotracheal tube without dependence on conventional adhesive systems alone that may fail on patients with facial hair or under high moisture conditions. More specifically, the mineral-based elastomer gel padding layers reduce risks of skin irritation, pressure injury, and formation of sores during prolonged intubation. The adjustable posterior and superior straps further distribute retention forces and enable customized fitting across varied patient anatomies and clinical positions. The translatable tube receiving housing further enhances positional control and reduces the likelihood of unintended tube migration or accidental extubation. Through these combined features, the device improves patient comfort, supports consistent airway management, and addresses limitations associated with existing stabilization devices.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an endotracheal tube holding device. The device stabilizes and retains an endotracheal tube relative to a patient while enhancing comfort and reducing displacement risks in conditions where adhesive holders demonstrate reduced effectiveness. Patient-contacting regions include a mineral-based elastomer gel interface configured to reduce skin irritation, pressure breakdown, shear forces, and the likelihood of accidental extubation or tube migration during prolonged use.

The device is comprised of a first strap that extends around the posterior head and neck regions to distribute retention forces and stabilize positioning. The first strap is comprised of elastic material, is adjustable via an adjustment fastener, and includes padding along a user-contacting surface. Opposing end regions of the first strap are coupled to cheek support members including a rear padding layer formed from mineral-based elastomer gel configured to releasably adhere to patient skin and conform to facial anatomy. A head strap is connected to the cheek support members and extends over a superior head region to provide vertical stabilization. The head strap is comprised of elastic material, is adjustable via an adjustment fastener, and includes padding along a user-contacting surface.

A connection member extends between the cheek support members across an upper lip region and supports a tube-receiving housing while maintaining spacing from nasal and oral structures. The connection member includes a rear padding layer and is rigid, semi-rigid, or flexible with an anatomically contoured profile. The tube-receiving housing is attached to the connection member and is configured for lateral translation to align with a user's mouth while maintaining engagement for continuous or incremental repositioning. The tube-receiving housing is comprised of a receiving port defining a through-opening configured to receive an endotracheal tube. An interior or exterior fastener secures the endotracheal tube relative to the tube-receiving housing, wherein in one embodiment the fastener is a ratcheting fastener configured to compress a portion of the receiving port around an outer surface of the endotracheal tube.

Accordingly, the endotracheal tube holding device of the present invention is particularly advantageous as it provides a device configured for secure stabilization of an endotracheal tube without dependence on conventional adhesive systems alone that may fail on patients with facial hair or under high moisture conditions. More specifically, the mineral-based elastomer gel padding layers reduce risks of skin irritation, pressure injury, and formation of sores during prolonged intubation. The adjustable posterior and superior straps further distribute retention forces and enable customized fitting across varied patient anatomies and clinical positions. The translatable tube receiving housing further enhances positional control and reduces the likelihood of unintended tube migration or accidental extubation. Through these combined features, the device improves patient comfort, supports consistent airway management, and overcomes the limitations associated with existing stabilization devices.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an endotracheal tube holding device of the present invention while worn by a user and while securing an endotracheal tube in accordance with the disclosed architecture;

FIG. 2 illustrates a perspective view of one potential embodiment of an endotracheal tube holding device of the present invention in accordance with the disclosed architecture;

FIG. 3 illustrates an enhanced perspective view of a connection member and tube-receiving housing of one potential embodiment of an endotracheal tube holding device of the present invention in accordance with the disclosed architecture; and

FIG. 4 illustrates an enhanced perspective view of a connection member and tube-receiving housing of one potential embodiment of an endotracheal tube holding device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that securely stabilizes an endotracheal tube. There also exists a long-felt need in the art for an endotracheal tube holding device that reduces skin irritation and pressure-related breakdown during prolonged use. Moreover, there exists a long-felt need in the art for an endotracheal tube holding device that accommodates patients with facial hair while maintaining adjustable positioning and comfort.

The present invention, in one exemplary embodiment, is comprised of an endotracheal tube holding device. The device stabilizes and retains an endotracheal tube relative to a patient while improving comfort and reducing displacement risks in environments where adhesive holders demonstrate reduced effectiveness. Patient-contacting regions are comprised of a mineral-based elastomer gel interface configured to reduce skin irritation, pressure injury, shear forces, and the likelihood of accidental extubation or tube migration during extended use.

The device is comprised of a first strap extending around the posterior head and neck regions to distribute retention forces and maintain positioning. The first strap is comprised of elastic material, is adjustable via an adjustment fastener, and includes padding along a user-contacting surface. Opposing end regions of the first strap are coupled to cheek support members including rear padding layers formed from mineral-based elastomer gel configured to releasably adhere to patient skin and conform to facial anatomy. A head strap is connected to the cheek support members and extends over a superior head region to provide vertical stabilization, wherein the head strap is comprised of elastic material, is adjustable, and includes padding.

A connection member extends between the cheek support members across an upper lip region to support a tube-receiving housing while maintaining spacing from nasal and oral structures. The connection member includes a rear padding layer and is rigid, semi-rigid, or flexible with an anatomically contoured profile. The tube-receiving housing is attached to the connection member and is configured for lateral translation to enable alignment with a user's mouth while maintaining engagement during continuous or incremental repositioning. The tube-receiving housing is comprised of a receiving port defining a through-opening configured to receive an endotracheal tube, and an interior or exterior fastener secures the endotracheal tube relative to the tube-receiving housing, wherein in one embodiment the fastener is a ratcheting fastener configured to compress a portion of the receiving port around an outer surface of the endotracheal tube.

As such, the device provides secure stabilization without reliance on adhesive systems alone and reduces risks associated with facial hair or high moisture conditions. Mineral-based elastomer gel padding layers reduce skin irritation and pressure injury during prolonged intubation, while adjustable posterior and superior straps distribute retention forces for customized fitting across varied anatomies and clinical positions. Lateral translation of the tube-receiving housing improves positional control and reduces the likelihood of unintended tube migration or accidental extubation, thereby enhancing comfort and supporting consistent airway management.

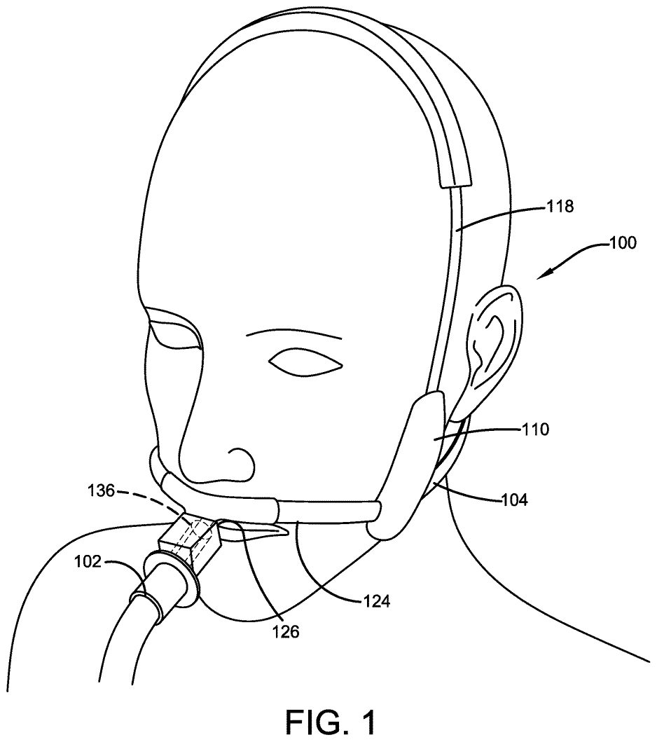

Referring initially to the drawings, FIG. 1 illustrates a perspective view of one potential embodiment of an endotracheal tube holding device 100 of the present invention while worn by a user and while securing an endotracheal tube 102 in accordance with the disclosed architecture. The device 100 is configured to stabilize and retain an endotracheal tube 102 relative to a patient while enhancing patient comfort and reducing risks associated with tube 102 displacement. More specifically, the device 100 may provide secure positioning of the endotracheal tube 102 in clinical environments where conventional adhesive-based tube 102 holders may experience reduced effectiveness, such as but not limited to patients with facial hair, high moisture conditions, facial movement, perspiration, or extended intubation durations. The device 100 may further be configured to reduce skin irritation, pressure-related breakdown, shear forces, and formation of sores through the use of a mineral-based elastomer gel interface positioned along patient-contacting regions. The device 100 may thereby assist in reducing the likelihood of accidental extubation, unintended tube migration, airway trauma, or loss of ventilatory support while enhancing overall patient comfort and stability during prolonged use.

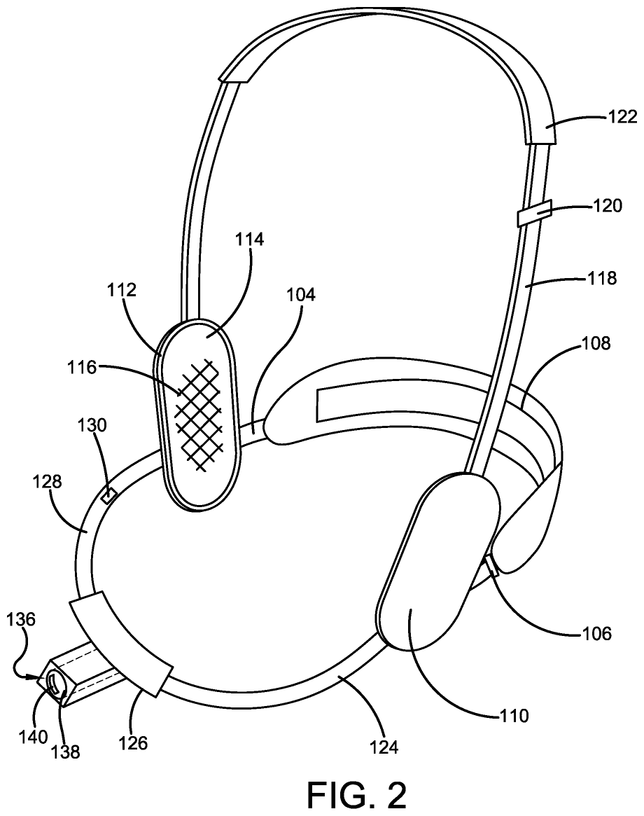

The device 100 is comprised of a first strap 104 configured to extend around a posterior region of a user's head and neck (as seen in FIG. 1) to stabilize the positioning of the device 100 during use. The first strap 104 may be dimensioned to extend across an occipital region, cervical region, or combinations thereof to provide distributed retention forces. In one embodiment, the first strap 104 may be comprised of an elastic material such as but not limited to woven elastomeric fabric, neoprene, rubberized textile, stretch laminate, knitted polymer band, etc. In another embodiment, the first strap 104 may be adjustable via an adjustment fastener 106 such as but not limited to a hook-and-loop fastener, buckle, sliding clasp, ratcheting strap interface, ladder lock, friction cinch, etc. The first strap 104 may further be comprised of a padding 108 (as seen in FIG. 2) positioned along at least a portion of a user-contacting surface of the strap 104 to enhance comfort and reduce localized pressure. The padding 108 may be continuous, segmented, removable, or selectively repositionable along the strap 104.

Each opposing end region of the first strap 104 may be coupled to a respective cheek support member 110. A rear surface 112 of each cheek support member 110 may be comprised of a padding layer 114 (as seen in FIG. 2) formed from a mineral-based elastomer gel configured to releasably adhere to a patient's skin. The mineral-based elastomer gel may be formulated to provide controlled tackiness, conformability, and shear resistance while allowing removal of the members 110 as needed. The padding layer 114 may have variable thickness profiles, contoured geometries, and in one embodiment, a surface texturing 116 to improve contact with facial anatomy such as zygomatic regions, buccal regions, or mandibular transition regions.

A head strap 118 may be connected to each cheek support member 110 and may be configured to extend over a superior region of a user's head (as seen in FIG. 1) to provide additional vertical stabilization of the device 100. The head strap 118 may be positioned centrally, offset, or angled relative to the cheek support members 110 to accommodate different head shapes or clinical positioning such as supine, lateral, or prone orientations. In one embodiment, the head strap 118 may be comprised of an elastic material such as but not limited to stretchable textile, braided polymer strand, laminate composite, elastomeric webbing, etc. In another embodiment, the head strap 118 may be adjustable via an adjustment fastener 120 such as but not limited to a cam lock, slider, ratchet assembly, cord lock, multi-position clasp, etc. The head strap 118 may further be comprised of a padding 122 positioned along a user-contacting surface, wherein the padding 122 may be tubular, planar, segmented, or integrated within the strap structure.

A connection member 124 may extend between the cheek support members 110 across a user's upper lip area, as seen in FIG. 1. The connection member 124 may provide a structural interface for positioning and supporting a tube-receiving housing 126 while maintaining spacing from the patient's nostrils and oral structures. A rear surface 128 of the connection member 124 may be comprised of a padding layer 130 (as seen in FIG. 2) such as but not limited to foam, gel, silicone cushion, microfiber textile, elastomer sleeve, etc. The connection member 124 may be rigid, semi-rigid, or flexible and may be comprised of materials such as but not limited to polymer, composite, metal, reinforced polymer, elastomer laminate, shape-retaining strip, etc. The connection member 124 may have a linear, curved, or anatomically contoured profile to follow facial curvature.

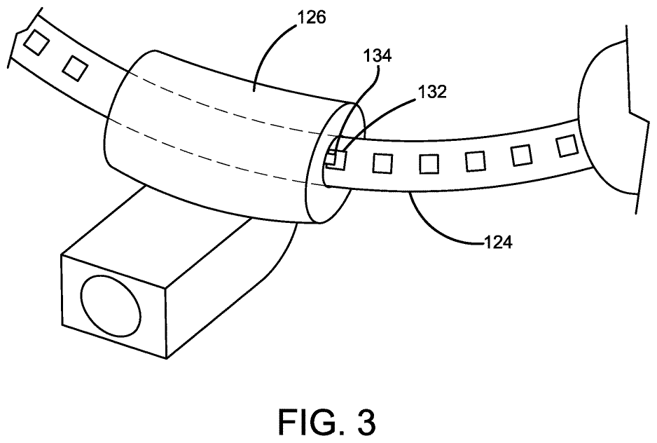

A tube-receiving housing 126 may be attached to the connection member 124 and may be configured for lateral translation along the connection member 124 to allow alignment with a user's mouth, as seen in FIG. 1. The tube-receiving housing 126 may translate continuously or incrementally and may maintain engagement with the connection member 124 during repositioning. In one embodiment, the tube-receiving housing 126 may be captured on the connection member 124 by an arrangement wherein the connection member 124 extends through the housing 126, as seen in FIG. 3. In a further embodiment, the connection member 124 may be comprised of fasteners 132 such as plurality of teeth, ridges, detents, serrations, or surface features that are receivable by complementary fasteners 134 (as seen in FIG. 3) such as but not limited to pawl elements, spring tabs, resilient fingers, friction pads, detent balls, elastomeric protrusions, compliant ribs, etc. within the housing 126 to permit incremental adjustment and positional retention of the housing 126 along the member 124.

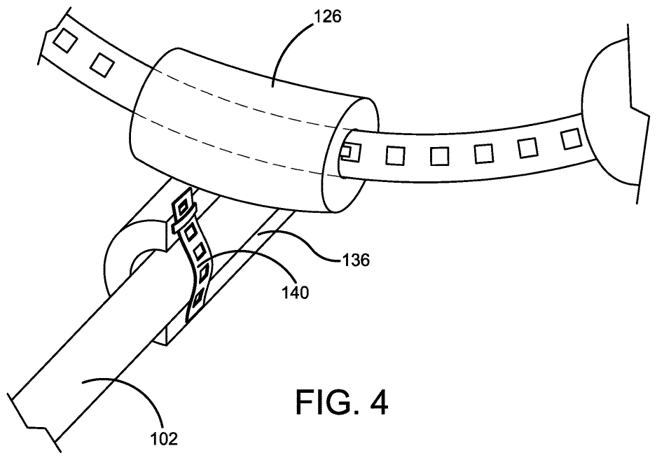

The tube-receiving housing 126 may be comprised of a receiving port 136 configured to receive an endotracheal tube 102, as seen in FIG. 1. In one embodiment, the receiving port 136 may be circular in cross-sectional shape to correspond with an outer circumference of the endotracheal tube 102 and may extend fully through the tube-receiving housing 126 to define a through-opening that allows insertion of the tube 102 from anterior or posterior directions. In another embodiment, the housing 126 is semi-circular in shape such that the housing 126 can be clipped onto and/or snap-fit onto the tube 102, as seen in FIG. 4. The receiving port 136 may have a constant diameter, tapered profile, stepped inner geometry, or elastically deformable structure to accommodate tubes of varying sizes. An interior surface 138 of the receiving port 136 may further be comprised of a fastener 140 such as but not limited to an adhesive layer, compressive sleeve, inflatable bladder, frictional insert, textured elastomer liner, high-friction coating, etc. to secure the tube 102 relative to the housing 126. In other embodiments, the fastener 140 may be positioned externally or internally on the receiving port 136 and may be but is not limited to clamping bands, ratcheting collars, pivoting jaws, hook-and-loop wraps, elastic constriction rings, compressive shells, etc. as seen in FIG. 4. In one embodiment, the fastener 140 may be a ratcheting fastener configured to compress at least a portion of the receiving port 136 around an outer surface of the endotracheal tube 102 to enhance retention and reduce unintended displacement.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “endotracheal tube holding device” and “device” are interchangeable and refer to the endotracheal tube holding device 100 of the present invention.

Notwithstanding the foregoing, the endotracheal tube holding device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the endotracheal tube holding device 100 as shown in the FIGS. are for illustrative purposes only, and that many other sizes and shapes of the endotracheal tube holding device 100 are well within the scope of the present disclosure. Although the dimensions of the endotracheal tube holding device 100 are important design parameters for user convenience, the endotracheal tube holding device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An endotracheal tube holding device comprising:
a first strap;
a first cheek support member attached to the first strap, wherein the first cheek support member is comprised of a first padding;
a second cheek support member attached to the first strap, wherein the first cheek support member is comprised of a second padding;
a second strap attached to the first cheek support member and the second cheek support member;
a connection member extending between the first cheek support member and the second cheek support member; and
a tube-receiving housing attached to the connection member further comprising a receiving area for an endotracheal tube.

2. The endotracheal tube holding device of claim 1, wherein the first padding and the second padding is an elastomer gel.

3. The endotracheal tube holding device of claim 2, wherein the elastomer gel is a mineral-based elastomer gel.

4. The endotracheal tube holding device of claim 1, wherein the first strap is an elastic strap.

5. The endotracheal tube holding device of claim 1, wherein the second strap is an elastic strap.

6. The endotracheal tube holding device of claim 1, wherein the connection member is comprised of a padding.

7. An endotracheal tube holding device comprising:
a first strap further comprising a first adjustment fastener;
a first cheek support member attached to the first strap, wherein the first cheek support member is comprised of a first padding;
a second cheek support member attached to the first strap, wherein the first cheek support member is comprised of a second padding;
a second strap attached to the first cheek support member and the second cheek support member, wherein the second strap member is further comprising a second adjustment fastener;
a connection member extending between the first cheek support member and the second cheek support member; and
a tube-receiving housing attached to the connection member further comprising a receiving area for an endotracheal tube.

8. The endotracheal tube holding device of claim 7, wherein the connection member is further comprised of a first fastener.

9. The endotracheal tube holding device of claim 8, wherein the tube-receiving housing is further comprised of a second fastener.

10. The endotracheal tube holding device of claim 7, wherein the connection member extends through the tube-receiving housing.

11. An endotracheal tube holding device comprising:
a first strap further comprising a first adjustment fastener;
a first cheek support member attached to the first strap, wherein the first cheek support member is comprised of a first padding;
a second cheek support member attached to the first strap, wherein the first cheek support member is comprised of a second padding;
a second strap attached to the first cheek support member and the second cheek support member, wherein the second strap member is further comprising a second adjustment fastener;
a connection member extending between the first cheek support member and the second cheek support member;
a tube-receiving housing attached to the connection member further comprising a receiving area for an endotracheal tube; and
a fastener positioned in the receiving area.

12. The endotracheal tube holding device of claim 11, wherein the fastener is positioned on an interior surface of the receiving area.

13. The endotracheal tube holding device of claim 11, wherein the receiving area is tapered.

14. The endotracheal tube holding device of claim 11, wherein the receiving area is elastically deformable.

15. The endotracheal tube holding device of claim 11, wherein the first padding and the second padding is an elastomer gel.

16. The endotracheal tube holding device of claim 15, wherein the elastomer gel is a mineral-based elastomer gel.

17. The endotracheal tube holding device of claim 11, wherein the first strap is an elastic strap.

18. The endotracheal tube holding device of claim 11, wherein the second strap is an elastic strap.

19. The endotracheal tube holding device of claim 11, wherein the first strap is comprised of a third padding that is repositionable along the first strap.

20. The endotracheal tube holding device of claim 11, wherein the second strap is comprised of a fourth padding that is repositionable along the second strap.
